(19) United States
(12) Patent Application Publication
(10) Pub. No.: US 2023/0389493 A1
(43) Pub. Date: Dec. 7, 2023

(54) METHOD AND APPARATUS FOR CULTIVATING SPROUT GINSENG USING AEROPONICS

(71) Applicant: MNTE CO., LTD., Incheon (KR)

(72) Inventors: Heung Sik KIM, Chungcheongnamdo (KR); Tae Cheol PARK, Incheon (KR)

(21) Appl. No.: 17/829,617

(22) Filed: Jun. 1, 2022

(51) Int. Cl. A01G 31/02; A23L 33/105; A61K 8/9789; A61K 36/258; A01G 7/06

(52) U.S. Cl. CPC A01G 31/02; A23L 33/105; A01G 7/06; A61K 36/258; A61K 8/9789

(57) ABSTRACT

A method for cultivating sprout ginseng includes a step of growing ginseng seedlings to sprout ginseng while ultrasonic-spraying a nutrient solution of a natural material Neomax to a root portion of the ginseng seedlings, where the nutrient solution of the natural material Neomax is a mixture of a natural material Neomax liquid and water, and the natural material Neomax liquid has the same ingredients and amounts (mg/L) as shown in Table 1 in the detailed description.

### BACKGROUND

The present invention relates to a method and an apparatus for cultivating sprout ginseng using aeroponics. In particular, the present invention is characterized in that functional ingredients contained in an extract are utilized by using a marine humus extract as a nutrient solution. In addition, the present invention is characterized in that a waste liquid is not generated by aeroculturing the nutrient solution to the ginseng seedling roots. Further, the present invention is characterized in that a light source is controlled to be optimized for ginseng cultivation. The method and the apparatus according to the present invention are characterized in that sprout ginseng can be cultivated in a very short period of time and is allowed to contain a large amount of rare ginsenoside ingredients.

Due to the outbreak of various viruses and air pollution around the world, researches on the pharmacological effects of rare ginsenoside ingredients contained in sprout ginseng, ginseng, red ginseng, ginseng fruit, and the like, which can be used as materials for pharmaceuticals, functional foods, cosmetics, and the like, have been actively conducted.

According to recent studies, ginsenoside, which has been known as a non-nutritive material, has various pharmacological effects such as anticancer, antioxidant, and cholesterol lowering.

However, despite these functional effects, ginseng is a very slow-growing crop that sprouts from the Rhizome once a year, forms leaves, and grows over several generations. It takes 3 to 4 years for the growth of fresh ginseng, and 5 to 6 years for the growth of medicinal ginseng and red ginseng, so that the recovery of capital is slow, which is very unfavorable to management.

In addition, ginseng is cultivated for a long time in the same soil and a fixed sun shade greenhouse for cultivation is open, resulting in suffering a lot of damage from diseases and pests, and thus chemical pesticides are frequently used for preventive control of the diseases and pests. The present applicant has devised a technique for cultivating sprout ginseng using ginseng seedlings to devise the present invention. According to this technology, the sprout ginseng can be cultivated and produced more than 8 times a year in indoor buildings or plastic tents, so that it is easy to be supplied economically. In addition, it is possible to obtain sprout ginseng and ginseng containing functional ginsenoside ingredients using ginseng seedlings grown for 30 days by an eco-friendly cultivation method.

Referring to prior art documents, “Water culture method of ginseng” is disclosed in Korean Patent Publication No. 10-1724400. In addition, “Producing method of ginseng seedlings and hydroponics of ginseng using the same” is disclosed in Korean Patent Publication No. 10-1806825. In the above documents, there is no description of a method such as using a marine humus extract as a nutrient solution or allowing the nutrient solution to be absorbed from the root portion by an ultrasonic spraying method, as in the present invention.

[Patent Documents] Korean Patent Publication No. 10-1724400 (registered on Apr. 3, 2017) and Korean Patent Publication No. 10-1806825 (registered on Dec. 4, 2017)

### SUMMARY OF THE INVENTION

It is an object of the present invention to provide a method for cultivating sprout ginseng that increases ginsenoside ingredients, and in particular contains rare ginsenoside ingredients even when cultivating a natural material Neomax by shortening a growth period of fresh ginseng by 30 days using a humidified aeroponics apparatus of an ultrasonic vibration device.

The present invention is directed to providing a method for cultivating sprout ginseng, including a step of growing ginseng seedlings to sprout ginseng while ultrasonic-spraying a nutrient solution of a natural material Neomax to a root portion of the ginseng seedlings,

wherein the nutrient solution of the natural material Neomax is a mixture of a natural material Neomax liquid and water, and

the ingredients of the natural material Neomax liquid have the same ingredients and amounts (mg/L) as shown in Table 1 below.

The nutrient solution of the natural material Neomax may be prepared by mixing water and the natural material Neomax liquid at a weight ratio of 200 to 750:1.

The sprout ginseng cultivated by the cultivation method

may contain 4.11 mg/g of ginsenoside Re and 0.30 mg/g of ginsenoside F1.

An apparatus for applying the cultivation method of claim 1, wherein when a nutrient solution device (26) of natural material Neomax supplemental water provides the nutrient solution through a pipe, the nutrient solution is sprayed as humidified mist from an ultrasonic vibration device (25), and the humidified mist (23) spreads in the lower space of a bed (21) through a blowing fan (24) like the humidified mist and is absorbed into the root of the ginseng seedling body so as to continuously absorb nutrients.

According to the present invention, one-year-old sprout ginseng produced by the method and the apparatus for cultivating the sprout ginseng using aeroponics is a new concept of functional agricultural product that is safe, contains a large amount of ginsenoside ingredients, and has reinforced rare ingredients, which has soft leaf stem, developed fine roots, strong fragrance, and high content of ginsenoside saponin in rhizomes, body, stems, leaves, etc. Accordingly, the sprout ginseng can be used for pharmaceuticals, cosmetics, and functional beverages.

In addition, there is a remarkable effect that one-year-old ginseng seedlings can be produced in a short period of 30 days using a nutrient solution prepared by diluting a natural material Neomax in water using the wavelength of an ultrasonic vibration device.

### BRIEF DESCRIPTION OF DRAWINGS

FIG. 1 is a flowchart showing a sequence of a method for cultivating ginseng using ultrasonic humidified aeroponics for producing a large amount of saponin ingredient.

FIG. 2 is a schematic diagram of an apparatus for implementing the cultivation method of FIG. 1.

FIG. 3 is a view showing a schematic diagram of FIG. 2 and a photograph of the actual cultivation together.

FIG. 4 is a photograph of sprout ginseng grown for 12 to 15 days after receiving a nutrient solution of the natural material Neomax to plant ginseng seedlings.

FIG. 5 is a photograph showing one-year-old sprout ginseng harvested in a state where the stem turns unusually dark purple and contains a large amount of ginsenoside ingredients after growing for 30 days.

FIG. 6 is a view capturing a result table requested of the analysis request as it is in the state of sprout ginseng harvested after growing one-year-old ginseng seedlings for 30 days by a cultivation method using a humidified aeroponic apparatus.

### DETAILED DESCRIPTION OF THE INVENTION

The features and advantages of the present invention will be more clearly understood by embodiments to be described by the accompanying drawings.

The applications of the present invention are not limited by the configuration and arrangement of components described in the embodiments of the present invention or illustrated in the drawings. The present invention can be implemented by other embodiments and performed by various methods. In addition, expressions and predicates used in the embodiments with respect to terms such as directions of devices or elements are used only to simplify the description of the present invention, and do not indicate or imply that the related devices or elements should simply have a specific direction. For example, in the case where terms such as “first” and “second” are used in the embodiments and claims describing the present invention, these terms are not intended to indicate or imply relative importance or purport.

In addition, the terms or words used in the present specification and claims should not be construed as being limited to conventional or dictionary meanings, and in order for the inventor to explain the terms and concepts of the invention in the best way, it should be interpreted as described based on the meaning and concept consistent with the technical idea of the present invention.

Therefore, the present invention is not limited to the presented embodiments, and various modifications and changes can be made by those skilled in the art to which the present invention pertains within the technical spirit of the present invention and an equivalent scope of the technical idea described in claims to be described below.

Although the present invention has been described based on a preferred embodiment with reference to the accompanying drawings, it will be apparent to those skilled in the art that various modifications can be made without departing from the technical scope thereof. Accordingly, the scope of the present invention may include a number of such modifications.

A humidified aeroponic method according to an embodiment of the present invention may be managed with economical maintenance costs because there is no generation of a waste nutrient solution in a cultivation tank. The existing method requires careful management because a nutrient solution discarded after using the nutrient solution during cultivation causes environmental pollution.

The present invention relates to a hydroponic bed method in which an ultrasonic nutrient solution supply device is installed for supplying a nutrient solution of a natural material Neomax so that humidified mist is introduced to the bottom of a bed by operating a blowing fan and sprayed around the roots of the ginseng seedling body, which is a method of supplying for 15 to 60 minutes and then stopping for 5 to 30 minutes, and which is a cultivation method that drastically reduces the nutrient solution comparative to a bottom-up and top-down aeroponics of a conventional mist nozzle method and does not generate contaminated waste liquid, reduces the supply amount of the nutrient solution, and increases the nutrient absorption efficiency in the root of ginseng seedlings, thereby increasing the content of a large amount of ginsenosides and thus having very high economic feasibility.

In addition, the present invention is a useful invention of providing a method and a system of cultivating ginseng using low-cost aeroponics, in which one-year-old sprout ginseng having a large amount of saponin ingredient is produced and increased by adjusting the time zone having the amount of more natural light comparative to a conventional control method by way of irradiating an artificial light source LED on the upper end of the cultivation tank according to the amount of natural light suitable for the growth of ginseng, the amount of light taking into account the amount of sleep required per day based on the time, and a time zone; and maintaining appropriate natural light and time zone required at all times in a control manner.

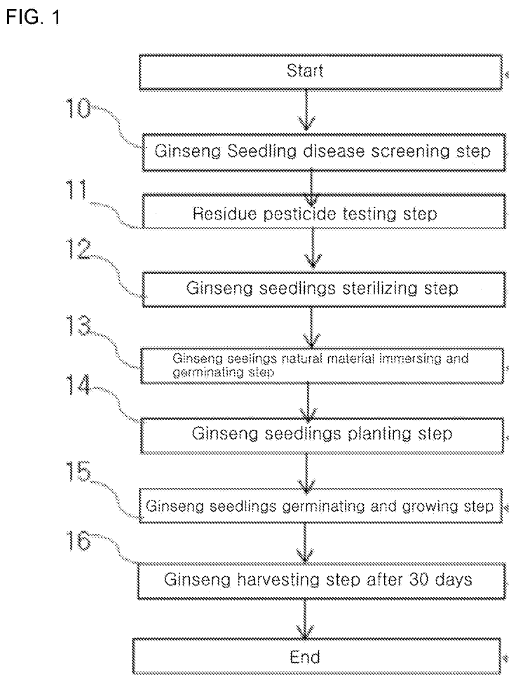

FIG. 1 is a flowchart showing a sequence of a method for cultivating ginseng using ultrasonic humidified aeroponics for producing a large amount of saponin ingredient.

The method includes performing visual screening of wound residual diseases of rhizome and the like of one-year-old ginseng seedlings that have been broken of rest (10), analyzing detection (GC-MS/MS and HPLC-MS/MS) by requesting a residual pesticide test of the visually screened ginseng seedlings through a quantitative analysis method (11), immersing and sterilizing the ginseng seedlings in purified water and chlorine dioxide water (500 to 1.00:1) before planting in the bed (12), absorbing nutrients and promoting germination by immersing the sterilized ginseng seedlings in a natural material Neomax under the conditions of temperature 17 to 20° C. and humidity 70 to 90% for 24 to 48 hours (13), planting the ginseng seedlings in grooves of a compressed polystylene bed plate at intervals of 100*75 mm (14), increasing a large amount of saponin ingredient in rhizomes, stems, leaves, etc. by germinating and growing the ginseng seedlings under artificial light conditions for 30 days while aero-spraying the nutrient solution ingredient of the natural material Neomax through ultrasonic humidification (15), and finally completing the harvest (16). Here, the natural material Neomax is an extract obtained by extracting mature marine soil (humus) using the technology of the present applicant, that is, an extract obtained by extracting the mature marine soil for 2 years after mining and fermenting the mature marine soil for 1 year.

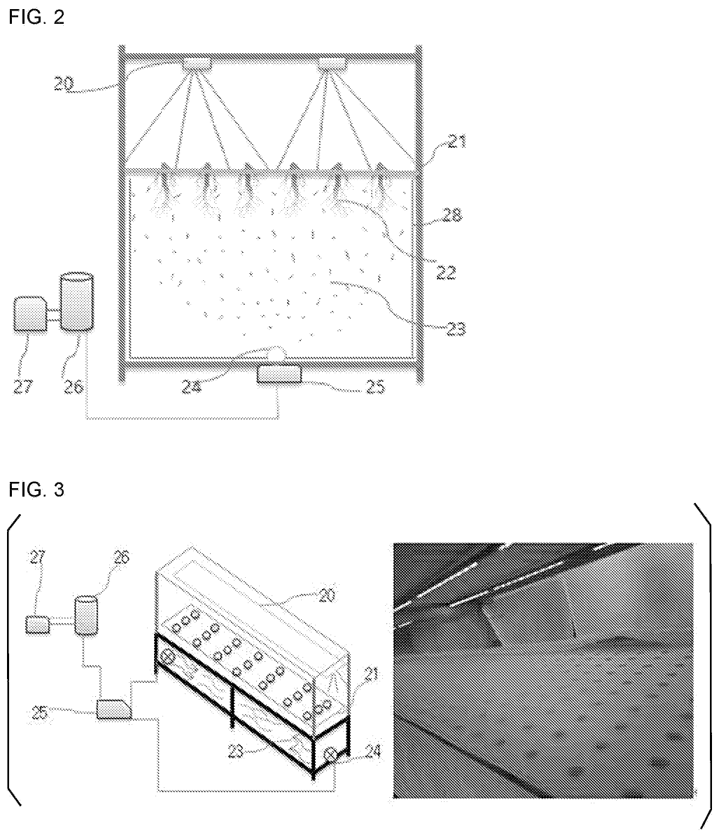

FIG. 2 is a schematic diagram of an apparatus for implementing the cultivation method of FIG. 1.

When a nutrient solution device 26 of natural material Neomax supplemental water provides the nutrient solution through a pipe, the nutrient solution is sprayed as humidified mist from the ultrasonic vibration device 25, and humidified mist 23 spreads in a lower space of a bed 21 through a blowing fan 24 like humidified mist and is absorbed into the root of the ginseng seedling body to continuously absorb nutrients, which is a method of supplying for 15 to 60 minutes and then stopping for 5 to 30 minutes.

Since the sprayed humidified mist does not generate any waste liquid, there is no need to collect the waste liquid, and thus no waste collection device is provided.

The brightness of a light source 20 varies depending on changes of time and temperature, and the illuminance is adjusted to 6.000 to 12.000 Lux from 5 a.m. before the natural sunrise of the sun until before sunset in the afternoon. At night after sunset, the brightness may be optimally controlled at 3.500 to 8.500 Lux until 02:00 the next day.

A controller 27 provides power and controls other components. The light source 20 may be configured with an LED.

FIG. 3 is a view showing a schematic diagram of FIG. 2 and a photograph of the actual cultivation together. As described above, the nutrient solution of the natural material Neomax is automatically supplied. The nutrient solution prepared by adding the natural material Neomax to raw water for supply is sprayed as the humidified mist to the ginseng seedlings by controlling a vibration wavelength useful for the physiological rhythm of ginseng seedlings with a predetermined ultrasonic frequency band.

Table 1 below shows mineral ingredients of the natural material Neomax used for aeroponics. (mg/L)

TABLE 1

| Ingredient | Content | Ingredient | Content | Ingredient | Content |
|---|---|---|---|---|---|
| Sodium | 91.00 | Potassium | 20.00 | Calcium | 240.00 |
| (Na) | | (K) | | (Ca) | |
| Magnesium | 92.00 | Sulfur (S) | 1.170.00 | Silicon | 38.00 |
| (Mg) | | | | (Si) | |
| Strontium | 2.00 | Iron (Fe) | 20.00 | Manganese | 6.80 |
| (Sr) | | | | (Mn) | |
| Aluminum | 330 | Zinc (Zn) | 3.10 | Yttrium | 0.40 |
| (Al) | | | | (Y) | |
| Nickel | 0.22 | Lithium | 0.30 | Copper | 0.62 |
| (Ni) | | (Li) | | (Cu) | |
| Verium | 0.10 | Selenium | 2.40 | Boron | 0.60 |
| (Be) | | (Se) | | (B) | |
| Cobalt | 0.20 | Lanthanum | Trace | Scandium | Trace |
| (Co) | | (La) | detection | (Sc) | detection |

Table 2 below shows amino acid components of the natural material Neomax used for aeroponics. (mg/100 g)

TABLE 2

| Ingredient | Content | Ingredient | Content | Ingredient | Content |
|---|---|---|---|---|---|
| Arginine | 71 | Lysine | 105 | Histidine | 33 |
| Phenylalanine | 90 | Tyrosine | 49 | Leucine | 117 |
| Isoleucine | 81 | Methionine | 24 | Valine | 123 |
| Alanine | 131 | Glycine | 136 | Poroline | 91 |
| Glutamic acid | 166 | Celine | 83 | Threonine | 93 |
| Aspartic acid | 184 | Tryptophan | 12 | Cystine | 52 |

The nutrient solution of the natural material Neomax is prepared with water: natural material Neomax at a ratio of 500 to 750:1 in a stage of 1 to 10 days, at a ratio of 300 to 500:1 in a stage of 11 to 20 days, and at a ratio of 200 to 300:1 in a 10 stage of 21 to 30 days (ratio: weight ratio) by planting early ginseng seedlings, and sprayed by setting pH to 5.5±5, and an ionic concentration to 0.6±0.1 dS/m in summer and 1.0±0.1 dS/m in winter.

When spraying with ultrasonic vibration, it is repeated to stop for 5 to 30 minutes after spraying for 15 to 60 minutes, and at this time the blowing fan supplies humidified mist by operating in the same manner.

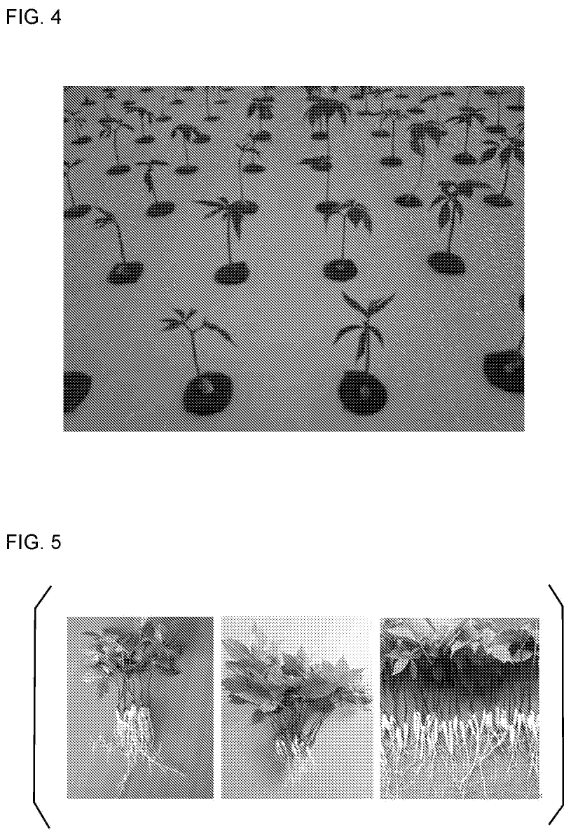

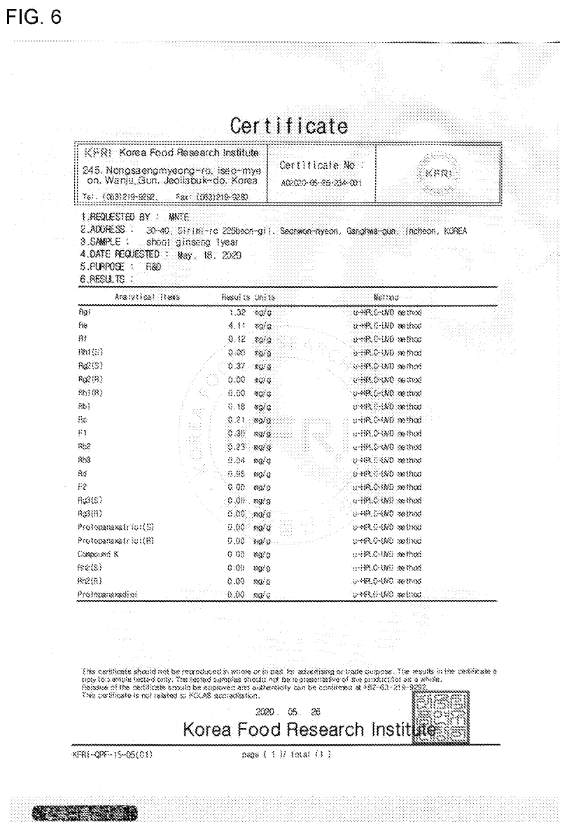

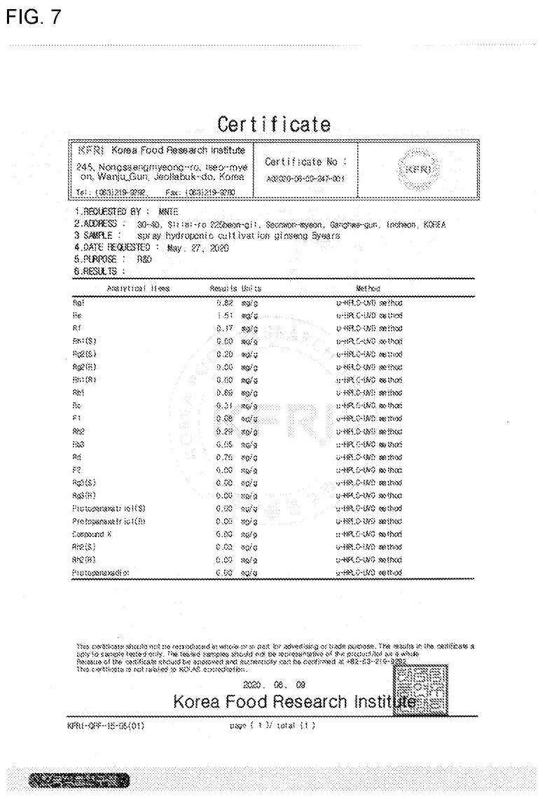

FIG. 4 is a photograph of sprout ginseng grown for 12 to 15 days after receiving a nutrient solution of the natural material Neomax and planting ginseng seedlings. FIG. 5 is a photograph showing one-year-old sprout ginseng harvested in a state where the stems turn unusually dark purple and a large amount of ginsenoside ingredients (FIG. 6) is contained after growing for 30 days.

FIG. 6 is a view capturing a result table of the analysis request as it is in the state of sprout ginseng harvested after growing one-year-old ginseng seedlings for 30 days by the cultivation method of the humidified aeroponic apparatus.

In detail, it can be seen that a large amount of ginsenoside ingredients is contained, and in particular it can be seen that a large amount of rare ginsenosides Re and F1 are contained.

FIG. 7 is a table showing ingredients of 5-year-old ginseng cultivated by another ginseng aeroponics system. The ginseng was cultivated by a 45-day hydroponic ginseng cultivation system that sprays a nutrient solution for ginseng cultivation on the market at regular intervals using a mist spray nozzle, not an aero-spraying method.

The content of the saponin ingredient was less than that of the ingredient analysis table of FIG. 6, and the rare ingredients did not appear even in the state of fresh ginseng.

Referring to FIG. 6, the content of the ginsenoside Re ingredient of fresh ginseng obtained by growing and harvesting ginseng seedlings for 30 days is 4.11 mg/g, which is 2.5 to 3 times higher than the content of Re 1.51 mg/g in a ginsenoside component analysis table (see FIG. 7) of 5-year-old fresh ginseng grown and harvested for 45 days of aeroponics.

In addition, FIG. 6 showed that the rare ginsenoside F1 ingredient is 0.30 mg/g, which is contained 4 times more than that of 5-year-old root.

According to the ingredient, 50% or more of Re is contained, and a large amount of ingredients Rg1, Rd, Rg2, and F1 are also contained. the ingredient Re serves main functions of an antidiabetic effect, insulin resistance reduction, a neuroprotective effect, an antioxidant effect, and a sperm fertility enhancement effect.

So far, the present invention has been described based on the preferred embodiment.

Therefore, the embodiments described in the present specification and the configurations illustrated in the drawings are merely the most preferred embodiment of the present invention and are not intended to represent all of the technical ideas of the present invention, and thus, it should be understood that there may be various modifications capable of replacing the embodiments.

Therefore, the present invention is not limited to the presented embodiments, and various modifications and changes can be made by those skilled in the art to which the present invention pertains within the technical spirit of the present invention and an equivalent scope of the technical idea described in the claims to be described below.

[Explanation of Reference Numerals and Symbols]

| | | |
|---|---|---|
| 20: Light source | 21: Bed | 22: Root |

| | |
|---|---|
| 23: Mist | 24: Blowing fan |

| | |
|---|---|
| 25: Ultrasonic vibration device | 26: Nutrient solution device of |

natural material Neomax supplemental water
27: Controller

1. A method for cultivating sprout ginseng comprising a step of growing ginseng seedlings to sprout ginseng while ultrasonic-spraying a nutrient solution of a natural material Neomax to a root portion of the ginseng seedlings,
wherein the nutrient solution of the natural material Neomax is a mixture of a natural material Neomax liquid and water, and
the natural material Neomax liquid has the same ingredients and amounts (mg/L) as shown in Table 1 in the detailed description.

2. The method for cultivating sprout ginseng of claim 1, wherein the nutrient solution of the natural material Neomax is prepared by mixing water and the natural material Neomax liquid at a weight ratio of 200 to 750:1.

3. The method for cultivating sprout ginseng of claim 1, wherein the sprout ginseng cultivated by the cultivation method contains 4.11 mg/g of ginsenoside Re and 0.30 mg/g of ginsenoside F1.

4. An apparatus for applying the cultivation method of claim 1, wherein when a nutrient solution device (26) of natural material Neomax supplemental water provides the nutrient solution through a pipe, the nutrient solution is sprayed as humidified mist from an ultrasonic vibration device (25), and the humidified mist (23) spreads in a lower space of a bed (21) through a blowing fan (24) like humidified mist and is absorbed into the root of the ginseng seedling body to continuously absorb nutrients.
